(19) United States
(12) Patent Application Publication
(10) Pub. No.: US 2012/0226041 A1
(43) Pub. Date: Sep. 6, 2012

(54) CATALYSTS AND BORONATE ESTERS

(75) Inventors: John F. Hartwig, Berkeley, CA (US); Carl W. Liskey, Oakland, CA (US)

(73) Assignee: The Board of Trustees of the University of Illinois, Urbana, IL (US)

(21) Appl. No.: 13/412,436

(22) Filed: Mar. 5, 2012

Related U.S. Application Data
(60) Provisional application No. 61/449,486, filed on Mar. 4, 2011.

(51) Int. Cl. C07F 5/04; C07F 19/00; C07D 493/08

(52) U.S. Cl. 546/10; 549/213; 558/288; 549/463; 546/2

(57) ABSTRACT

The invention provides catalysts and methods for preparing boronate esters. The methods can include the borylation of a secondary or primary C—H bond, for example, by contacting a reactant having a methylene or methyl and a diboron bis-ester in the presence of an effective catalyst. The contacting can be in the presence of an iridium complex, to effect borylation of the secondary or primary C—H bond, thereby providing the boronate ester. The boronate esters can be readily isolated and/or converted into other useful compounds and intermediates.

### RELATED APPLICATIONS

This application claims priority under 35 U.S.C. §119(e) to U.S. Provisional Patent Application No. 61/449,486, filed Mar. 4, 2011, which is incorporated herein by reference.

### GOVERNMENT SUPPORT

This invention was made with government support under Grant No. CHE 0910641 awarded by the National Science Foundation. The United States Government has certain rights in the invention.

### BACKGROUND OF THE INVENTION

The borylation of aromatic or aliphatic C—H bonds generates synthetically valuable organoboron compounds. Complementary to Friedel-Crafts reactions or chelation-assisted C—H functionalizations, the borylation of aromatic or aliphatic C—H bonds occurs at the least sterically hindered position. Organoboron compounds are versatile synthetic intermediates that can be converted into a variety of organic compounds through standard synthetic transformations.

Iridium catalysts have been used to perform the C—H borylation of arenes, and rhodium and ruthenium catalysts have been used in the borylation of primary C—H bonds. The rhodium and ruthenium catalysts complexes react exclusively with primary C—H bonds and no functionalization is observed with secondary C—H bonds. Accordingly, new synthetic methods are needed for the borylation of primary and secondary C—H bonds. Also needed are new catalysts for the borylation of primary and secondary C—H bonds.

### SUMMARY

The invention provides new catalysts to conduct the borylation of secondary aliphatic C—H bonds and new methods for borylation of aliphatic methylene and methyl groups. The catalyst can be, for example, an iridium complex having an optionally substituted phenanthroline ligand. In some embodiments, the iridium complex can be an iridium(III) complex. In various embodiments, iridium complexes can be prepared from diboron bis-esters such as bispinacolatodiboron, resulting in the inclusion of pinacolatoboron ligands on the iridium.

Accordingly, the invention provides methods to borylate a secondary or primary C—H bond comprising contacting a reactant that includes an aliphatic hydrocarbon moiety having a methylene or methyl, and a diboron bis-ester in the presence of an effective amount of an iridium complex for a period of time sufficient to effect borylation of the secondary or primary C—H bond, to provide a product that includes a boronate ester. The iridium complex can include a ligand having two sp²-hybridized nitrogen atoms that act as electron donors to the iridium of the complex. For example, the iridium complex can include an optionally substituted phenanthroline ligand or an optionally substituted dihydroimidazolyl-pyridine ligand. In some embodiments, the iridium complex includes a tetramethylphenanthroline ligand, a phenanthroline ligand, or a 2-(1-methyl-4,5-dihydro-1H-imidazol-2-yl)pyridine ligand.

The iridium complex can be used in about 0.1 mol % to about 50 mol %, about 1 mol % to about 20 mol %, about 2 mol % about 15 mol %, or about 5 mol % to about 10 mol %, with respect to the reactant. In some embodiments, the iridium complex can be used in less than about 30 mol %, less than about 20 mol %, less than about 15 mol %, less than about 10 mol %, or less than about 5 mol %, with respect to the reactant.

In some embodiments, the diboron bis-ester used to prepare the catalyst is bispinacolatodiboron. In other embodiments, a dioxaborolane can be used to prepare the catalyst.

A variety of reactants can be borylated using the methods described herein. For example, the reactant can be an optionally substituted cyclic ether. The cyclic ether can be, for example, an optionally substituted 4, 5, 6, 7, or 8 membered ring. In some embodiments, the reactant includes a substituted cyclopropane. For example, the substituted cyclopropane can include an alkyl, aryl, substituted aryl, halo, nitrile, or carboxy ester, or a combination thereof.

The method of the invention can further include isolating the boronate ester by chromatography, and/or converting the boronate ester to other useful compounds such as a secondary alcohol or a secondary alkylarene.

The invention further provides methods to synthesize a boronate ester that includes a bond between boron and a saturated carbon atom comprising contacting a reactant having a methylene or methyl, and a diboron bis-ester or dioxaborolane in the presence of an iridium complex for a period of time sufficient to effect borylation of a secondary or primary C—H bond of the methylene or methyl, to provide the boronate ester product.

The invention additionally provides methods to synthesize a boronate ester containing a bond between boron and a saturated carbon atom comprising contacting a cyclic ether or a substituted cyclopropane, and bispinacolatodiboron (or equivalent boron containing group), in the presence of an iridium complex having a tetramethylphenanthroline ligand. The contacting can be carried out at a temperature of about 30° C. to about 150° C., optionally in a suitable organic solvent, for a period of time sufficient to effect borylation of a secondary C—H bond of the cyclic ether or substituted cyclopropane, to provide the boronate ester. In some embodiments, the method can exclude solvents, thereby carrying out the reaction as a ‘neat’ mixture.

In some embodiments, the iridium complex can be formed from bis-pinacolatodiboron (B₂pin₂) or a dioxaborolane, an optionally substituted bidentate Lewis base compound such as a phenanthroline, a bipyridine, or an N-methyl imidazolyl-pyridine, and [Ir(*═*)OR]₂, wherein *═* is an alkene-containing moiety such as cyclooctene or cyclooctadiene and R is an alkyl or aryl group such as methyl or phenyl. In various embodiments, the iridium complex can be formed from bis-pinacolatodiboron (B₂pin₂) or a dioxaborolane, tetramethylphenanthroline (tmphen), and [Ir(COD)OMe]₂ or a similar iridium complex. In other embodiments, the iridium complex can be formed from tetramethylphenanthroline (tmphen) and (η⁶-mes)Ir(Bpin)₃, or a similar iridium complex.

In some embodiments, the iridium catalyst can be an iridium complexes of formula X:

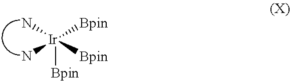

where the bidentate nitrogen ligand (—N—N—) is phenanthroline, tetramethylphenanthroline (tmphen), or 2-(1-methyl-4,5-dihydro-1H-imidazol-2-yl)pyridine, and Bpin is a pinacolatoborane ligand.

Accordingly, the invention provides novel catalyst complexes and useful intermediates for the synthesis of various compounds, as well as methods of preparing such compounds using the methods described herein.

### BRIEF DESCRIPTION OF THE DRAWINGS

The drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention, however, one skilled in the art will understand that portions of the example or aspect may be used in combination with other examples or aspects of the invention.

FIG. 1 illustrates the catalytic cycle of Ir-catalyzed C—H borylation of arenes, where R is an arene moiety of a molecule and the H of the R—H is bonded to an aryl carbon. The C—H borylation of alkane moieties, where R is an alkane moiety of a molecule and the H of the R—H is bonded to a primary or secondary carbon, is believed to follow an analogous catalytic cycle.

### DETAILED DESCRIPTION

Pentamethylcyclopentyldienyl (Cp*) rhodium and ruthenium complexes are known to catalyze the C—H borylation of primary C—H bonds. These complexes are unreactive toward the borylation of secondary C—H bonds. Iridium complexes containing a 4,4′-di-tert-butyl-2,2′-bipyridine (dtbpy) ligand catalyze the C—H borylation of arenes. These complexes do not catalyze the borylation of secondary C—H bonds.

The new iridium complexes described herein successfully perform the borylation of secondary and primary C—H bonds. The iridium complex can be, for example, an iridium(III) complex. In some embodiments, the iridium complex can include a bidentate Lewis base such as tetramethylphenanthroline as an ancillary ligand. The combination of an iridium catalyst and bispinacolatodiboron allows for the borylation of secondary C—H bonds of cyclopropanes. The combination of an iridium catalyst, tetramethylphenanthroline, and bispinacolatodiboron allows for the borylation of secondary C—H bonds of a cyclic ether or N-protected pyrrolidine. For borylation of N-protected pyrrolidines such as pivalate-protected pyrrolidines, elevated temperatures, such as about 100-150° C., or about 130-140° C., can be advantageous.

Accordingly, described herein are methods to replace a hydrogen atom of an aliphatic hydrocarbon moiety with a boronate ester (B(OR)₂) group. The invention thus provides methods for performing C—H borylation of secondary alkyl C—H bonds, in addition to other substrates such as primary C—H bonds and aryl C—H bonds. The organoboron compounds that are formed in these reactions are synthetically versatile intermediates that can be converted to a variety of organic compounds through known functional group transformations.

The methods described herein thus provide new processes for the functionalization of secondary alkyl C—H bonds. Specifically, the methods can be used to form secondary alkylboronate esters. The methods allow for the direct conversion of compounds containing secondary and primary C—H bonds to the corresponding organoboron compound. The products generated in these reactions are generally stable and can be isolated via traditional techniques, including silica-gel chromatography.

A number of cyclic ethers and substituted cyclopropanes undergo conversion to the corresponding organoboron compounds in the presence of, for example, bis-pinacolatodiboron (B₂pin₂) and the combination of iridium and tetramethylphenanthroline as an ancillary ligand. For example, the borylation of tetrahydrofuran can be performed in the presence of an iridium complex, a tetramethyl-phenanthroline ligand, and bis-pinacolatodiboron. Primary C—H bonds can also undergo the borylation reaction.

The alkylboronate esters generated in these reactions can be readily converted to secondary alcohols and to secondary alkylarenes using standard synthetic transformations. The secondary alkylboronate esters can also be further derivatized to provide scaffolds for combinatorial libraries. For example, boronic acids or esters can be transformed into myriad functional groups including aryl or vinyl groups via Suzuki-Miyaura couplings (Miyaura and Suzuki, Chem. Rev. 95: 2457-2483 (1995); Suzuki, J. Organomet. Chem. 576: 147-168 (1999); Miyaura, In Advances in Metal-Organic Chemistry, Liebeskind, Ed.: JAI: London, Vol. 6, pp. 187-243 (1998); see also Metal-catalyzed Cross-coupling Reactions; Diederich and Stang, Eds.: Wiley: Wienheim, 1998). Organoboron compounds can also undergo efficient transmetallation to palladium and other transition metals followed by reactions with aryl halides and the like, or coupling under oxidative conditions, to provide various synthetically valuable compounds. Numerous examples of such reactions are described in standard references texts such as March's Advanced Organic Chemistry: Reactions, Mechanisms, and Structure, 5^{th} Ed. (M. B. Smith and J. March, John Wiley & Sons, New York, 2001).

The invention thus provides methods to borylate a secondary or primary C—H bond and the products of such reactions. The method can include contacting a reactant having a methylene or methyl, and a diboron bis-ester or diolate-substituted borane typically called a dioxaborolane, in the presence of an iridium complex, for a period of time sufficient to effect borylation of the secondary or primary C—H bond, to provide a product having a boronate ester substituent.

The iridium complex can include a ligand having two sp²-hybridized nitrogens, such as an optionally substituted phenanthroline ligand or an optionally substituted dihydroimidazolyl-pyridine ligand. Examples of such ligands include, but are not limited to, tetramethylphenanthroline, 4,7-dimethoxyphenanthroline, 2,9-dimethylphenanthroline, phenanthroline, or 2-(1-methyl-4,5-dihydro-1H-imidazol-2-yl)pyridine.

The iridium complex can be used in a stoichiometric amount or in a catalytic amount. For example, the methods described herein can be carried out wherein the iridium complex is present in less than about 50 mol %, less than about 25 mol %, less than about 20 mol %, less than about 10 mol %, less than about 8 mol %, less than about 5 mol %, less than about 2 mol %, or less than about 1 mol %, with respect to the reactant. Similar amounts, including amounts 1-5 mol % greater in each instance, can be used for the molar amount of the iridium ligands. The iridium complex can be formed, for example, from a diboron bis-ester or dioxaborolane and [Ir(COD)OMe]₂ or (η⁶-mes)Ir(Bpin)₃. In one embodiment, the diboron bis-ester is bispinacolatodiboron (B₂pin₂). In another embodiment, the dioxaborolane is pinacolborane (HBpin)

The reactant can be a molecule that includes a methyl or methylene C—H bond. Examples include cyclic ethers, such as an optionally substituted 4, 5, 6, 7, or 8 membered ring, and alkanes, such as octane. The reactant can also be a substituted cyclopropane or an optionally substituted 4-8 membered cycloalkane. The substitutions of the cyclopropane or other cycloalkane can include one or more alkyl, aryl, substituted aryl, halo, nitrile, ketone, amide, secondary amine, tertiary amine, or carboxy ester substituents, as well as ether and/or amide linkages (within the cycloalkane ring, or on or as substituents), or a combination thereof. Examples of suitable substrates and reaction conditions include those illustrated in Schemes 4 and 5 below. The substrates and reaction conditions can be varied to provide other products, as would be readily understood by one of skill in the art. Generally the borylation will not be effective on reactants that include alkene, aldehyde, free hydroxyl, or free amino groups. However, other than alkenes, aldehydes, free hydroxyls, and free amino groups, any combination of the functional groups described earlier in this paragraph will be well tolerated by the borylation reaction.

The boronate esters can be isolated by any suitable method, including chromatography, such as silica gel chromatography. The methods can further include converting the boronate ester to a secondary alcohol (e.g., by hydrolysis) or a secondary alkylarene (e.g., by coupling with an aryl halide using a palladium catalyst), and/or any other suitable synthetic transformation of boronate esters.

In one embodiment, the invention provides a method to synthesize an aliphatic boronate ester comprising contacting a reactant having a methylene or methyl, and a diboron bis-ester or dioxaborolane in the presence of an iridium complex for a period of time sufficient to effect borylation of a secondary or primary C—H bond of the methylene or methyl, to provide the boronate ester. In another embodiment, the invention provides a method to synthesize an aliphatic boronate ester comprising contacting a cyclic ether or a substituted cyclopropane, and bispinacolatodiboron or pinacolborane in the presence of an iridium(III) complex having a tetramethylphenanthroline ligand, at a temperature of about 30° C. to about 150° C., optionally in a suitable organic solvent, for a period of time sufficient to effect borylation of a secondary C—H bond of the cyclic ether or substituted cyclopropane, to provide the boronate ester.

### Iridium Catalysts

New catalysts that can be used to conduct the borylation of secondary aliphatic C—H bonds, such as aliphatic methylene and methyl groups, include iridium complexes having an optionally substituted bidentate Lewis base compound such as a phenanthroline, a bipyridine, or an N-methyl imidazolyl-pyridine. Such ligands may or may not have symmetric or asymmetric substitution such as hydrogen atoms, linear or branched C₁₋₈ alkyl groups, linear or branched C₁₋₈ alkoxy groups, nitro groups, cyano groups, halogenated C₁₋₈ alkyl groups, halogen atoms, carbamoyl groups, C₁₋₈ acyl group, C₁₋₈ alkoxycarbonyl groups, or amino groups, which may or may not have further substituents. The amount of such ligands used in a reaction can be about 0.01 mol % to about 50 mol %, about 0.1 mol % to about 20 mol %, or about 1 mol % to about 10 mol %, with respect to the compound having the secondary aliphatic C—H bond. In some embodiments, the amount of the bidentate Lewis base can be about twice or about thrice the amount of the mol % of iridium used in the reaction. In one embodiment, the optionally substituted bidentate Lewis base can be a phenanthroline ligand.

A ligand on the iridium complex can be carbon monoxide or an alkene-containing compound. Such alkene-containing compounds can be, for example, COD (1,5-cyclooctadiene), COE (1-cyclooctene) or indene. The carbon monoxide or alkene-containing compound can dissociate from the iridium to provide the active catalyst.

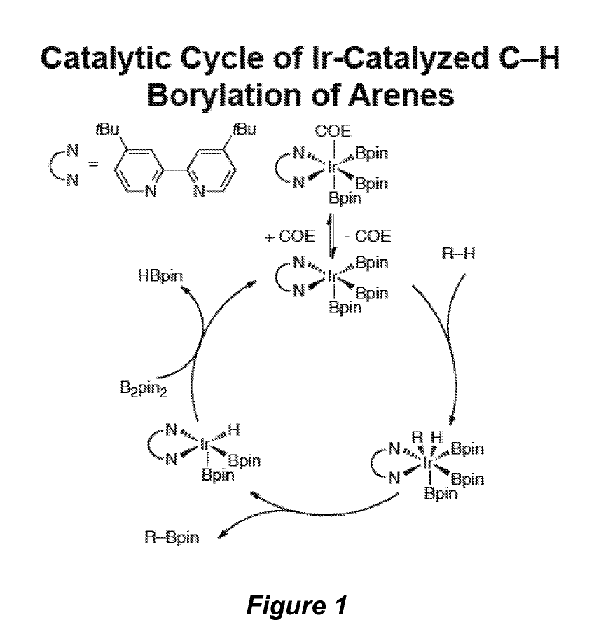

In some embodiments, the iridium complex can be an iridium(III) complex. In various embodiments, iridium complexes can be prepared from diboron bis-esters such as bispinacolatodiboron or dioxaborolanes such as pinacolborane or a catecholborane such as 4-tert-butylcatechol-borane, resulting in the inclusion of a boron-containing ligands on the iridium. Other boron moieties that can be used to prepare useful iridium catalysts are described in U.S. Pat. No. 6,878,830 (Smith). In one embodiment, the iridium catalyst is a complex of formula X:

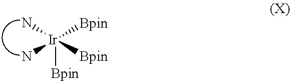

where the bidentate nitrogen ligand is, for example, a phenanthroline, a bipyridine, or an N-methyl imidazolyl-pyridine. In some specific embodiments, the bidentate nitrogen ligand is an optionally substituted phenanthroline such as tetramethylphenanthroline (tmphen) or 2-(1-methyl-4,5-dihydro-1H-imidazol-2-yl)pyridine.
Secondary Aliphatic C—H Bond Borylation A catalytic cycle of Ir-catalyzed C—H borylation of arenes is illustrated in FIG. 1. Ir-trisboryl complexes are active catalysts for the C—H borylation of arenes. These complexes contain neutral bidentate ligands. The development of a catalyst for the borylation of aliphatic C—H bonds analogous to the highly active arene borylation catalyst is described herein.

A number of iridium-trisboryl complexes containing different substituents on boron and ancillary ligands were prepared to investigate the reactivity and electronic properties. The reactivity of Ir complexes was investigated to identify features that lead to highly active catalysts for C—H borylation reactions. As indicated by Scheme 1 below, the Ir (Bpin)₃ complex reacted faster and formed products in higher yields than Ir(Bcat*)₃.

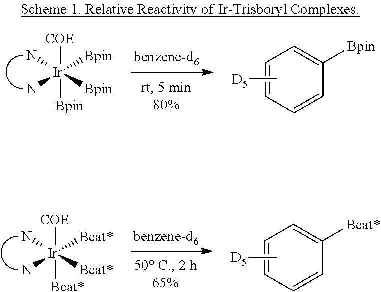

The reaction of the phosphine-ligated iridium complex required higher temperatures and longer reaction times to proceed to only 33% conversion (Scheme 2). The phosphine-ligated iridium complex also does not bind COE, potentially due to the enhanced steric properties of the bulky phosphine ligand.

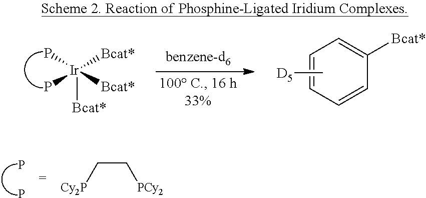

The electronic properties of Ir-trisboryl complexes were also investigated.

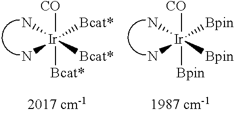

IR spectral analysis of the Ir(Bcat*)₃ complex and the Ir(Bpin)₃ complex showed peaks at 2017 cm⁻¹ and 1987 cm ⁻¹, respectively. These data are consistent with greater donation of electron density from the more electron-rich tris(Bpin)-complexes into the π* anti-bonding orbital of the olefin and carbonyl ligands. It was determined that electron-rich and nonsterically bulky ligands can lead to more active complexes for aliphatic borylation.

Strongly electron-donating and nonsterically demanding ligands were evaluated for the borylation of aliphatic C—H bonds. As shown in Scheme 3, reactions with tetramethylphenanthroline as the ligand gave high yields of the alkylboronate ester products.

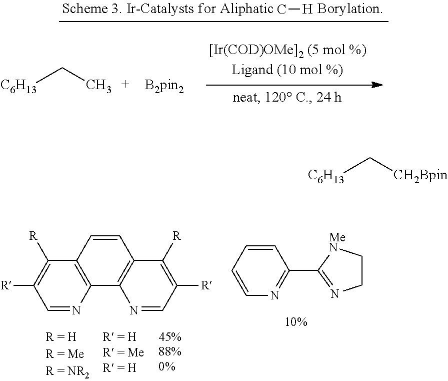

Using tetramethylphenanthroline as a ligand in an Iridium complex, C—H borylation of a methylene carbon was achieved for the first time. In several examples, cyclopropanes containing alkyl, electron poor and rich aryl, cyano, and bromo substituents were converted to cyclopropylboronate esters in good yields (Scheme 4).

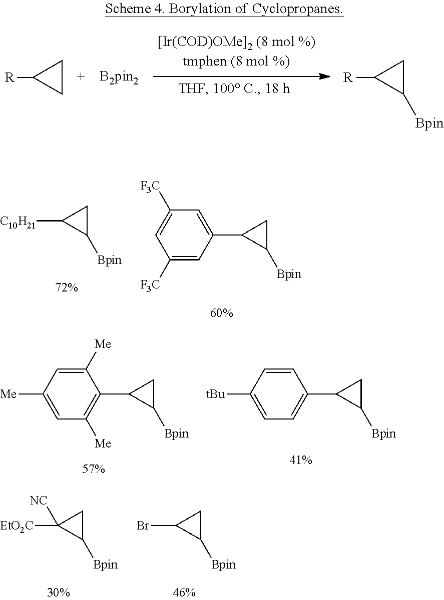

As shown in Scheme 5, cyclic ethers having 4-, 5-, and 6-membered rings were converted to the corresponding borylated product in good yields under neat conditions in only 18 hours.

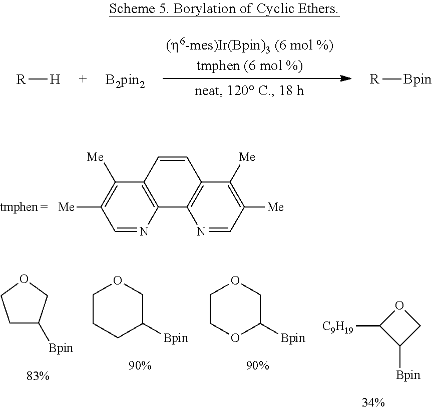

Two pathways in which the C—H activation can occur are illustrated in Scheme 6.

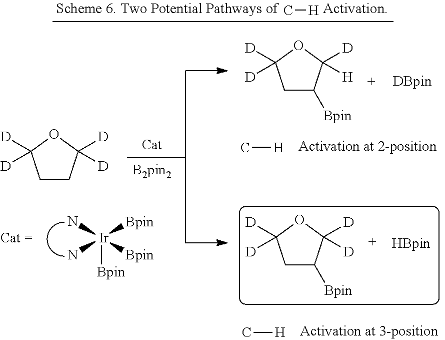

The d₄ product was formed exclusively and only HBpin was observed by ¹¹B NMR, thus the C—H activation was shown to have occurred exclusively at the site of borylation. Accordingly, the C—H borylation of secondary C—H bonds has been successfully accomplished, and new catalysts to perform the borylation of secondary C—H bonds are described herein.

### Definitions

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is disubstituted.

The term “about” can refer to a variation off ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer. Unless indicated otherwise herein, the term “about” is intended to include values, e.g., weight percents, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible subranges and combinations of subranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into subranges as discussed above. In the same manner, all ratios recited herein also include all subratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution or in any reaction mixture, including a ‘neat’ mixture of reactants.

The term “effective amount” can refer to an amount of a compound described herein, or an amount of a combination of compounds described herein, that is effective to promote or cause a chemical reaction to occur, such as a catalytic reaction. Thus, an “effective amount” generally means an amount that provides the desired effect.

The term “iridium complex” refers to an inorganic or organometallic complex with at least one iridium atom and one or more ligands associated with the iridium. The iridium of the complex can have a variety of oxidation states. The active catalytic state of an iridium catalyst for borylation can be (III). Many iridium(III) complexes can be prepared from iridium(I) complexes, and the iridium may pass through an oxidation state of (V) during a borylation catalytic cycle.

The term “hydrocarbon moiety” refers to a section of a reactant molecule that includes only carbon and hydrogen atoms such as a reactant with a methylene C—H bond. The hydrocarbon moiety can be 1°, 2°, 3°, or 4°, but the borylation reactions described herein are carried out on a secondary or primary carbon, such as a methylene group or a methyl group. The reactant that includes the hydrocarbon moiety (e.g., a reactant in a borylation reaction as described herein) can be an exclusively hydrocarbon molecule, or the reactant can include heteroatoms and/or various functional groups, such as the reactants shown in Schemes 4 and 5.

The term “borylate” or “borylation” refers to modifying a carbon-hydrogen bond (or other carbon-“leaving group” bond) to provide a carbon-boron bond.

The term “bis(pinacolato)diboron” (B₂pin₂) refers to the diborane compound having the structure

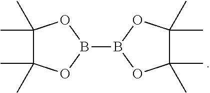

B₂pin₂ can be used to prepare useful iridium complexes; however other diolate-substituted boranes can also be used in place of B₂pin₂ for preparing the catalysts and carrying out the methods described herein. Examples of other effective boranes for preparing iridium catalysts and carrying out the methods described herein include derivatives of B₂pin₂ and dioxaborolanes such as pinacolborane (HBpin), 4-tert-butylcatechol-borane, hexyleneglycolato diborons, and various borane compounds. Examples of such useful boron reagents are further described in U.S. Pat. No. 6,451,937 (Hartwig et al.).

The following abbreviations are used in this application.

Bcat* refers to “4-tert-butylcatecholboryl” and HBcat* refers to a 4-tert-butylcatecholborane ligand or moiety.

Bpin refers to “pinacolatoboron”.

CO refers to “carbon monoxide”.

COD refers to “cyclooctadiene”.

COE refers to “cyclooctene”.

η⁶-mes refers to “hexahapto mesitylene” or a six-coordinate mesitylene ligand.

The abbreviation tmphen refers to “tetramethylphenanthroline”.

### General Preparatory Methods

The catalytic methods described herein can use any of the applicable techniques of organic synthesis and the related arts. Many such techniques are well known to the skilled artisan. Accordingly, many of the known techniques are elaborated in, for example, Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith; as well as March, J., Advanced Organic Chemistry, Third Edition, (John Wiley & Sons, New York, 1985); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999).

Additional information and useful techniques known to those of skill in the art are described by U.S. Pat. No. 6,451,937 (Hartwig et al.) and the following publications: Murphy, J. M., Lawrence, J. D., Kawamura, K., Incarvito, C., and J. F. Hartwig, Ruthenium-Catalyzed Regiospecific Borylation of Methyl C—H Bonds. J. Am. Chem. Soc., 2006. 13684-13685; Chen, H. Y., Schlecht, S., Semple, T. C., and J. F. Hartwig, Thermal, catalytic, regiospecific functionalization of alkanes. Science, 2000. 287(5460): 1995-1997; and Ishiyama, T., Takagi, J., Ishida, K., Miyaura, N., Anasrasi, N. R., and J. F. Hartwig, Mild iridium catalyzed borylation of arenes. High turnover numbers, room temperature reactions, and isolation of a potential intermediate. J. Am. Chem. Soc., 2002. 124(3): 390-391.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

The complex [Ir(cod)OMe]₂ was obtained from Johnson-Matthey, and B₂pin₂ was obtained from Allychem Co., Ltd.

### Example 1

### Secondary Borylation Reactions

The scope of the borylation reaction was evaluated, in part, by performing a series of reactions on cyclic ethers and cycloalkanes. Results of various reactions are shown below in Table 1. The borylation reactions generally proceeded smoothly as neat reaction mixtures, nearing completion in 14 hours in many cases.

General Procedure for the Borylation of Cyclic Ethers.

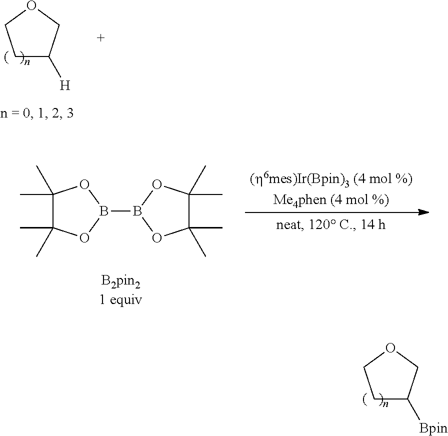

In a nitrogen-filled glove box, B₂pin₂ (127 mg, 0.500 mmol), (η⁶-mesitylene)Ir(Bpin)₃ (14 mg, 0.020 mmol), and tetramethylphenanthroline (4.7 mg, 0.020 mmol) were combined in a 4 mL vial with a stirbar. The substrate (0.5 mL) was added and the vial was sealed with a Teflon-lined cap. The reaction was heated to 120° C. for 14-18 h in a heating block. The solution became dark red upon heating. The completion of the reaction was monitored by gas chromatography. The volatile materials were evaporated under reduced pressure and the crude boronate ester was isolated by column chromatography on silica gel with a gradient of 100:0 to 85:15 pentane:Et₂O.

TABLE 1
Examples of Borylation Substrates and Products.

| Entry | Reactant | Product | Yield (%)^{a} |
|---|---|---|---|
| 1 | 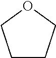 | 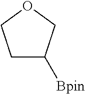 | 83 |
| 2 | 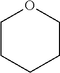 | 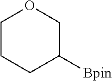 | 90 |
| 3 | 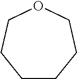 | 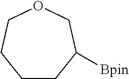 | 65^{b} |
| 4 | 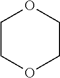 | 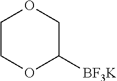 | 64^{c} |
| 5 | 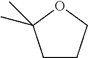 | 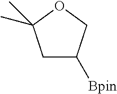 | 61 |
| 6 | 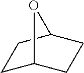 | 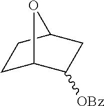 | 63^{d} |
| 7 | 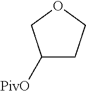 | 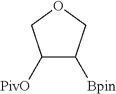 | 45^{b} |
| 8 | 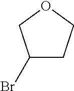 | 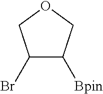 | 35^{b} |
| 9 | 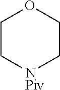 | 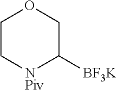 | 41^{c} |
| 10 | 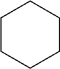 | 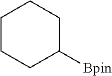 | 39^{e} |
| 11 | 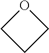 | 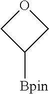 | 25^{b} |

^{a}Yield of secondary boronate ester product isolated by silica-gel chromatography.
^{b}Yield by ¹¹B NMR.
^{c}Yield of isolated product following conversion to the corresponding trifluoroborate salt.
^{d}Yield of isolated product following conversion to the benzoate protected alcohol.
^{e}Reaction conducted at 140° C. with 10% Ir catalyst, yield determined by gas chromatography.

### Product Characterization Data.

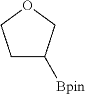

Entry 1 Product. ¹H NMR (500 MHz, CDCl3) δ 3.99 (t, J=8.3 Hz, 1H), 3.80 (td, J=8.1, 4.1 Hz, 1H), 3.70 (dt, J=8.0, 6.9 Hz, 1H), 3.61 (dd, J=9.7, 8.2 Hz, 1H), 2.10-1.96 (m, 1H), 1.88-1.75 (m, 1H), 1.67-1.54 (m, 1H), 1.24 (s, 12H). ¹³C NMR (126 MHz, CDCl3) δ 8 83.69, 70.59, 68.78, 29.06, 25.06. Anal. Calc'd for CHN: C, 60.64; H, 9.67; N, 0.00. Found C, 60.56%; H, 9.95%; N, 0.02.

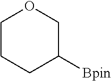

Entry 2 Product. ¹H NMR (500 MHz, CDCl3) δ 3.86 (m, 2H), 3.48 (m, 2H), 1.83 (d, J=8.9 Hz, 1H), 1.60-1.53 (m, 2H), 1.38-1.27 (m, 1H), 1.26 (s, 12H). ¹³C NMR (126 MHz, CDCl3) δ 83.45, 70.11, 68.93, 27.09, 25.34, 25.13, 25.07. HRMS Calc'd 213.1662. Found 213.1665.

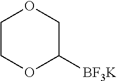

Entry 3 Product. ¹H NMR (500 MHz, DMSO) δ 3.52-3.24 (m, 6H), 2.72-2.59 (m, 1H). ¹³C NMR (126 MHz, DMSO) δ 66.5, 63.6, 63.1. HRMS calc'd 232.9765. Found 232.9773.

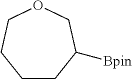

Entry 4 Product. GC-MS m/z=226. ¹¹B NMR: δ 33.6 ppm.

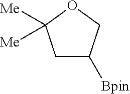

Entry 5 Product. ¹H NMR (400 MHz, CDCl3) δ 4.06-3.95 (m, 1H), 3.75 (dt, J=10.4, 7.0 Hz, 1H), 1.93-1.75 (m, 2H), 1.69 (d, J=9.9 Hz, 1H), 1.27 (s, 3H), 1.24 (s, 12H), 1.18 (s, 3H). ¹³C NMR (101 MHz, CDCl3) δ 83.34, 80.77, 69.28, 41.32, 28.09, 27.79, 24.75. ¹¹B NMR 33.4.

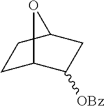

Entry 6 Product. ¹H NMR (600 MHz, CDCl3) δ 8.05 (d, J=7.2 Hz, 2H), 7.55 (t, J=7.4 Hz, 1H), 7.43 (t, J=7.8 Hz, 2H), 5.02 (dd, J=7.2, 2.5 Hz, 1H), 4.71 (t, J=5.1 Hz, 1H), 4.67 (d, J=5.9 Hz, 1H), 2.08 (dd, J=13.3, 7.2 Hz, 1H), 1.92-1.86 (m, 1H),1.83-1.68 (m, 2H), 1.54-1.48 (m, 1H), 1.48-1.41 (m, 1H).

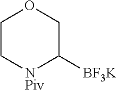

Entry 7 Product. ‘H NMR (400 MHz, DMSO) δ 4.08 (dd, J=17.1, 9.5 Hz, 1H), 3.93 (d, J=13.6 Hz, 1H), 3.67 (dd, J=11.1, 2.6 Hz, 1H), 3.11 (td, J=11.6, 2.5 Hz, 1H), 2.75 (dt, J=22.4, 10.8 Hz, 2H), 2.42 (d, J=11.6 Hz, 1H), 1.15 (d, J=3.3 Hz, 9H).

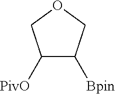

Entry 8 Product. GC-MS: m/z=283 (m/z -Me group). ¹¹B NMR: δ 33.9.

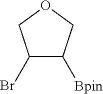

Entry 9 Product. GC-MS: m/z 278, 276. ¹¹B NMR: δ 33.6.

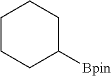

Entry 10 Product. GC-MS: m/z 210. ¹¹B NMR: δ 33 ppm.

### Example 2

### Cyclopropane Borylation Reactions

The scope of the borylation reaction was further evaluated by performing a series of reactions on various cyclopropane compounds. Results of various reactions are shown below in Table 2. The borylation reactions generally proceeded smoothly in THF, nearing completion in 18 hours in many cases.

General Procedure for the Borylation of Cyclopropanes.

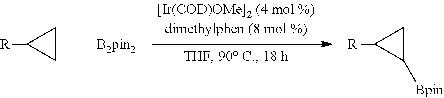

In a nitrogen-filled glove box, B₂pin₂ (127 mg, 0.500 mmol), [Ir(COD)OMe]₂ (26 mg, 0.020 mmol), and 2,9-dimethylphenanthroline (8.3 mg, 0.040 mmol) were combined in a 4 mL vial with a stirbar and dissolved in tetrahydrofuran (0.5 mL). The substrate (0.60 mmol) was added and the vial was sealed with a Teflon-lined cap. The reaction was heated to 90° C. for 18 h in a heating block. The solution became dark red upon heating. The completion of the reaction was monitored by gas chromatography. The volatile materials were evaporated under reduced pressure and the crude boronate ester was isolated by column chromatography on silica gel with a gradient of 100:0 to 80:20 pentane:Et₂O.

TABLE 2
Examples of Borylation Substrates and Products.

| Entry | Product | Yield (%) | d.r.^{a} |
|---|---|---|---|
| 1 | 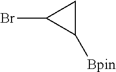 | 73 | 12:1 |
| 2 | 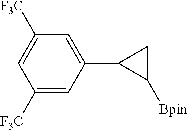 | 60 | n.d. |
| 3 | 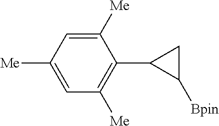 | 57 | n.d. |
| 4 | 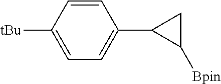 | 41 | 4:1 |
| 5 | 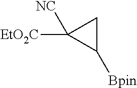 | 30 | >15:1 |
| 6 | 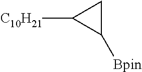 | 72 | n.d. |
| 7 | 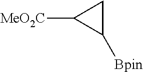 | 65 | 11:1 |
| 8 | 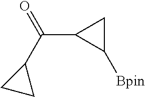 | 65 | 10:1 |

^{a}diastereoselectivity determined by GC of the crude reaction mixture.
n.d. = not determined.

Product Characterization Data.

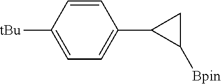

Entry 1 Product. ¹H NMR (499 MHz, CDCl₃) δ 7.27 (d, 2H), 7.03 (d 2H), 2.10 (dt, 1H), 1.31 (s, 9H), 1.26 (s, 12H) 1.23 (m, 1H), 1.11 (m, 1H), 0.94 (m, 1H), 0.23 (m, 1H). GC-MS: m/z =285 (-Me group).

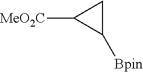

Entry 2 Product. ¹H NMR (400 MHz, CDCl₃) δ 3.70 (s, 3H), 1.79 (tt, 1H), 1.26 (overlapping peak, 1H) 1.25 (s, 12H), 0.97 (m, 1H), 0.61 (ddd, 1H). GC-MS: m/z=226.

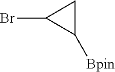

Entry 3 Product. GC-MS m/z=233, 231 (m/z -Me group). ¹¹B NMR=33.5 ppm.

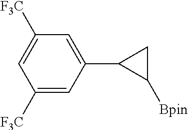

Entry 4 Product. GC-MS: m/z=325 (-Me group).

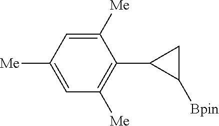

Entry 5 Product. GC-MS : m/z=271 (-Me group).

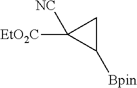

Entry 5 Product. GC-MS m/z=250 (-Me group). ¹¹B NMR 33.5 ppm.

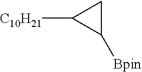

Entry 7 Product. GC-MS m/z=293 (-Me group). ¹¹B NMR 33.6 ppm.

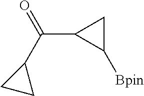

Entry 8 Product. GC-MS m/z=236. ¹¹B NMR 33.7 ppm.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method to borylate a secondary C—H bond comprising:
contacting a reactant having a methylene group, and a diboron bis-ester or dioxaborolane in the presence of an effective amount of an iridium complex for a period of time sufficient to effect borylation of the secondary C—H bond, to provide a product that includes a boronate ester.

2. The method of claim 1 wherein the iridium complex comprises a ligand having two sp²-hybridized nitrogen atoms that act as electron donors to the iridium of the complex.

3. The method of claim 2 wherein the iridium complex comprises an optionally substituted phenanthroline ligand or an optionally substituted dihydroimidazolyl-pyridine ligand.

4. The method claim 3 wherein the iridium complex comprises a tetramethylphenanthroline ligand, a phenanthroline ligand, or a 2-(1-methyl-4,5-dihydro-1H-imidazol-2-yl)pyridine ligand.

5. The method claim 1 wherein the iridium complex is present in less than about 20 mol % with respect to the reactant.

6. The method of claim 1 wherein the diboron bis-ester is bispinacolatodiboron.

7. The method claim 4 wherein the reactant comprises a cyclic ether.

8. The method of claim 7 wherein the cyclic ether comprises an optionally substituted 4, 5, 6, 7, or 8 membered ring.

9. The method of claim 1 wherein the reactant comprises a substituted cyclopropane.

10. The method of claim 9 wherein the substituted cyclopropane comprises an alkyl, aryl, substituted aryl, halo, nitrile, or carboxy ester, or a combination thereof.

11. The method of claim 1 further comprising isolating the boronate ester by chromatography.

12. The method of claim 1 further comprising converting the boronate ester to a secondary alcohol or a secondary alkylarene.

13. A method to synthesize a boronate ester that includes a bond between boron and a saturated carbon atom comprising:
contacting a reactant having a methylene group, and a diboron bis-ester or dioxaborolane in the presence of an iridium complex for a period of time sufficient to effect borylation of a secondary C—H bond of the methylene group, to provide the boronate ester product.

14. A method to synthesize a boronate ester containing a bond between boron and a saturated carbon atom comprising:
contacting a cyclic ether or a substituted cyclopropane, and bispinacolatodiboron, in the presence of an iridium complex having a tetramethylphenanthroline ligand, at a temperature of about 30° C. to about 150° C., optionally in a suitable organic solvent, for a period of time sufficient to effect borylation of a secondary C—H bond of the cyclic ether or substituted cyclopropane, to provide the boronate ester.

15. The method of claim 1 wherein the iridium complex is formed from bis-pinacolato-diboron and [Ir(COD)OMe]₂ or (η⁶-mes)Ir(Bpin)₃.

16. An iridium catalyst of formula X: 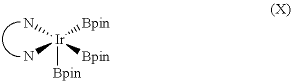
where the bidentate nitrogen ligand is phenanthroline, tetramethylphenanthroline (tmphen), or 2-(1-methyl-4,5-dihydro-1H-imidazol-2-yl)pyridine, and Bpin is a pinacolatoborane ligand.
